(19) United States
(12) Patent Application Publication
(10) Pub. No.: US 2019/0160093 A1
(43) Pub. Date: May 30, 2019

(54) THERAPEUTIC AGENT FOR DISEASE ACCOMPANIED BY EPILEPTIC WAVES

(71) Applicant: TAIYO NIPPON SANSO CORPORATION, Tokyo (JP)

(72) Inventors: Mamoru Ouchida, Okayama-shi (JP); Iori Ohmori, Okayama-shi (JP); Yuko Kaida, Kawachinagano-shi (JP); Hirotsugu Takeuchi, Tokyo (JP); Mitsuhiro Ogawa, Tokyo (JP); Toshie Furuumi, Tokyo (JP)

(21) Appl. No.: 16/265,173

(22) Filed: Feb. 1, 2019

Related U.S. Application Data
(63) Continuation of application No. 14/772,473, filed on Jan. 22, 2016, now abandoned.

(30) Foreign Application Priority Data
Jul. 22, 2010 (JP) 2010-164770

(51) Int. Cl. A61K 33/00; A61M 15/00; A61K 9/00; A61M 16/00

(52) U.S. Cl. CPC A61K 33/00; A61M 15/00; A61M 2202/0225; A61M 16/0045; A61K 9/0073; A61K 9/007

(57) ABSTRACT

Provided is an inhalation gas device for a therapy of a disease accompanied by epileptiform discharges comprising a medical gas bottle, and a medical inhalation gas device connected to the medical gas bottle, the inhalation gas device for a therapy of a disease accompanied by epileptiform discharges including the following 1) and 2):
1) a therapeutic agent for a disease accompanied by epileptiform discharges containing carbon dioxide as an active ingredient being filled in the medical gas bottle; and
2) the medical inhalation gas device being provided with a gas inhalation mask.

### CROSS REFERENCE TO RELATED APPLICATIONS

This Application is a Continuation-in-part of U.S. patent application Ser. No. 14/772,473 filed on Jan. 22, 2016, which application is a 371 U.S. National Phase Application of International PCT Patent Application No. PCT/JP2011/065845, filed Jul. 12, 2011, which application claims priority to Japanese Patent Application No. JP 2010-164770 filed on Jul. 22, 2010. The entire contents of these applications are incorporated herein by reference in their entirety.

### TECHNICAL FIELD

The present invention relates to a therapeutic agent for a disease accompanied by epileptiform discharges, characterized by containing carbon dioxide as an active ingredient. In addition, the present invention relates to a medical device for administering a therapeutic agent for a disease accompanied by epileptiform discharges, containing carbon dioxide as an active ingredient.

The present application makes a request for priority based on the Japanese Patent Application No. 2010-164770, which is incorporated herein by reference.

### BACKGROUND ART

In Japan, it is said that about one million epileptic patients are present (announced by Japan Neurosurgical Society and the Japan Epilepsy Society). Epilepsy means paroxysmal or repetitive abnormalities in muscle contraction, consciousness, perception, behavioral movement, or an autonomic nervous system, caused by sudden abnormal excitability of nerve cells in the brain, and is characterized by a chronic recurrent seizure caused by pathological lesions in the brain. In epilepsy, epileptiform discharges are detected in electroencephalograms in many cases. However, for example, when no seizure occurs, epileptiform discharges are not necessarily detected. Meanwhile, even when epileptiform discharges are detected in electroencephalograms, some cases are not diagnosed as epilepsy.

The prevalence of epilepsy is about from eight to ten people per one thousand people. Epilepsy is a disease having a high frequency in neurological diseases. Examples of a main symptom of epilepsy include convulsive seizure (involuntary movement such as tonic seizure or clonic seizure) and an absence seizure (loss of consciousness) without a convulsive component. In recent years, causative genes of epilepsy have been identified by the development of molecular genetics study. As a result, a relationship between epilepsy and various channel genes has become evident. It has come to be considered that some epilepsy syndromes are so-called channelopathy caused by mutations in channel genes. Particularly, mutations of a voltage-dependent Ca²⁺ channel, a voltage-dependent Na⁺ channel or K⁺ channel, and the like have been detected in epileptic patients.

As for a current therapeutic agent for epilepsy, a synthetic compound is administered orally, a suppository is administered, or administration is performed intravenously. Examples of the current therapeutic agent for epileptic patients include an oral administration agent of a synthetic compound such as a channel blocker. Particularly in status epilepticus, a fast-acting medication is desired. Therefore, intravenous injection of a medical substance is performed. However, there is a problem due to at least (1) to (3) described below. (1) There is a drug-resistant epilepsy syndrome, and in some cases, an effect is still insufficient. (2) In status epilepticus, it is necessary to administer a medical substance intravenously, while this treatment cannot be performed to a patient whose intravenous line is not easily ensured (such as children). (3) In a case of an unpredictable seizure on admission, it is possible to administer a medical substance intravenously. However, in a case of an unpredictable seizure at home or the like, it is not possible to administer a medical substance intravenously, and oral administration takes too much time before an effect thereof appears, therefore prolonged convulsive seizure results in increasing the risk of a sequela. In a case of an unpredictable seizure, a hospitalized patient can be treated by intravenous administration. However, in a case of non-admission, a fast-acting medication (prior art) does not exist. This is because oral administration or a suppository takes much time before effects thereof appear.

As an anti-epileptic drug which can be used when a sufficient effect cannot be obtained with other epileptic drugs, acetazolamide (AZA) is known (Non Patent Literature 1). Acetazolamide is a carbonic anhydrase inhibitor and inhibits a carbonic anhydrase in a process of producing water and carbon dioxide from carbonic acid. It is known that, as a result, excretion of sodium bicarbonate is increased and metabolic acidosis is thereby caused. However, acetazolamide is an internal medicine, and therefore has no fast-acting property against an unpredictable seizure outside a hospital. In addition, there is a risk of lowering a blood pH excessively by administration thereof. Furthermore, as an important side effect, a central nervous symptom such as shock, anemia (aplastic anemia, hemolytic anemia, agranulocytosis, thrombocytopenic purpura), mucocutaneous ocular syndrome, toxic epidermal necrosis, acute renal failure, renal-urinary tract stone, mental confusion, or convulsive seizure, may appear. Acetazolamide also has a problem that it takes too much time before an effect thereof appears.

Therefore, it is desired to develop a therapeutic agent for an epileptic seizure, which is easily handled, has a low side effect, and has a fast-acting property. It has been reported that when 8-11 days-old rats are placed at 48±2° C. for 55 minutes, alkalosis in the brain triggers febrile seizures and that alkalosis in the brain can be suppressed by placing the rats in a chamber containing 5% carbon dioxide (Non Patent Literature 2). However, there is no report of study of an effect of carbon dioxide on epilepsy. It is considered that febrile seizures are caused by a complex combination of a genetic factor, a degree of body temperature, and an immaturity of neuron. However, this report (Non Patent Literature 2) does not consider the genetic factor having the largest importance.

### CITATION LIST

### Non Patent Literatures

Non Patent Literature 1: Br Med J., Mar. 24, 1956, 650-654
Non Patent Literature 2: Nature Medicine, 12, 817-823 (2006)

### SUMMARY OF INVENTION

### Technical Problem

An object of the present invention is to provide a therapeutic agent for a disease accompanied by epileptiform discharges, which is easily handled, has a low side effect, and has a fast-acting property.

### Solution to Problem

The present inventors made intensive studies using epileptic model rats in order to solve the above problems. As a result, the present inventors have succeeded in changing the blood pH to an acidic side to be able to suppress epileptiform discharges by making the model rats inhale carbon dioxide and controlling the concentration of carbon dioxide of inhaled air, and have completed the present invention.

That is, the present invention includes the following.

1. An inhalation gas device for a therapy of a disease accompanied by epileptiform discharges comprising a medical gas bottle, and a medical inhalation gas device connected to the medical gas bottle, the inhalation gas device for a therapy of a disease accompanied by epileptiform discharges including the following 1) and 2):

1) a therapeutic agent for a disease accompanied by epileptiform discharges containing carbon dioxide as an active ingredient being filled in the medical gas bottle; and

2) the medical inhalation gas device being provided with a gas inhalation mask.

2. The inhalation gas device for a therapy of a disease accompanied by epileptiform discharges according to item 1, wherein the medical inhalation gas device of 2) includes a gas extraction member which is attached to an opening portion of the medical gas bottle of 1) and discharges the therapeutic agent for a disease filled in the medical gas bottle from a discharge nozzle.
3. The inhalation gas device for a therapy of a disease accompanied by epileptiform discharges according to item 2, wherein the gas inhalation mask provided in the medical inhalation gas device of 2) is a gas inhalation mask which has a base side connecting portion in which a mask base portion side is connected to the discharge nozzle of the medical inhalation gas device of 2), an inhalation shape portion provided on a mask distal end side having a shape in which a distal end edge covers a periphery of a mouth and a nose of an inhaler when used, and an enlarged portion which connects the base side connecting portion and the inhalation shape portion.
4. The inhalation gas device for a therapy of a disease according to item 3, wherein the entire gas inhalation mask is formed of a soft material through which no gas permeates, and a peripheral edge gas introducing portion is provided on a distal end portion peripheral edge of the inhalation shape portion, the peripheral edge gas introducing portion expanding by introduction of the therapeutic agent for a disease discharged from the discharge nozzle to have a shape of the inhalation shape portion, and being contractible when the therapeutic agent for a disease is not introduced,

a resistance member which gives resistance against flow of the therapeutic agent for a disease from the discharge nozzle toward the inhalation shape portion is provided on the base side connecting portion, and

a gas guide flow path, which introduces the therapeutic agent for a disease discharged from the discharge nozzle into the peripheral edge gas introducing portion, branches from a space between the resistance member and the discharge nozzle in the base side connecting portion.

5. The inhalation gas device for a therapy of a disease according to item 4, wherein the resistance member is a sintered filter, an orifice, or a valve.
6. The inhalation gas device for a therapy of a disease according to item 4, wherein the gas guide flow path is integrally formed with the enlarged portion.
7. The inhalation gas device for a therapy of a disease according to item 3, wherein the entire gas inhalation mask is formed of a soft material through which no gas permeates, and a peripheral edge gas introducing portion is provided on a distal end portion peripheral edge of the inhalation shape portion, the peripheral edge gas introducing portion expanding by introduction of the therapeutic agent for a disease discharged from the discharge nozzle to have a shape of the inhalation shape portion, and being contractible when the therapeutic agent for a disease is not introduced,

a shielding member which shields flow of the therapeutic agent for a disease from the discharge nozzle toward the inhalation shape portion is provided in the base side connecting portion,

a gas guide flow path, which introduces the therapeutic agent for a disease discharged from the discharge nozzle into the peripheral edge gas introducing portion, branches from a space between the shielding member and the discharge nozzle in the base side connecting portion, and an inhalation gas discharge portion which discharges the therapeutic agent for a disease toward inside of the enlarged portion is provided in at least one of the peripheral edge gas introducing portion and the gas guide flow path.

8. The inhalation gas device for a therapy of a disease according to item 7, wherein the gas guide flow path is integrally formed with the enlarged portion.
9. The inhalation gas device for a therapy of a disease according to item 1, wherein the disease accompanied by epileptiform discharges is epilepsy.
10. The inhalation gas device for a therapy of a disease according to item 1, wherein the therapeutic agent for a disease accompanied by epileptiform discharges containing carbon dioxide as an active ingredient is an inhalation therapeutic agent.
11. The inhalation gas device for a therapy of a disease according to item 10, wherein the medical gas bottle is filled with a therapeutic agent for a disease so that a concentration of carbon dioxide in the inhaled gas at the time of inhalation is 1 to 10% (v/v).

### Advantageous Effects of Invention

According to the therapeutic agent of the present invention for a disease accompanied by epileptiform discharges, containing carbon dioxide as an active ingredient, it is observed that a spike duration caused by epileptiform discharges is suppressed. Therefore, the therapeutic agent suppresses epileptiform discharges by inhalation of carbon dioxide to a disease accompanied by epileptiform discharges, and can act effectively.

In addition, by development of a gas bottle or an inhalation gas device using the therapeutic agent of the present invention for a disease accompanied by epileptiform discharges, the following effects are expected.

(1) An effect of suppressing a seizure is fast-acting.

(2) In a case of an unpredictable seizure at home or the like, surrounding persons (family members or the like) can cope therewith easily.

(3) Use thereof is simple.

(4) It is inexpensive.

(5) In status epilepticus, it is necessary to administer a medical substance intravenously. A fast-acting medication can be performed to a patient whose intravenous line is not easily ensured (such as children).

(6) When an epilepsy syndrome which is not easily suppressed with conventional medical substances is treated or status epilepticus is treated in an intensive care unit, it is possible to suppress a seizure while breathing is completely controlled.

### BRIEF DESCRIPTION OF DRAWINGS

FIG. 1 illustrates a brain in which an electrode for measuring electroencephalograms of an epileptic model rat in the present invention is embedded. (Examples 1 and 4, Comparative Example 1, Reference Example 1)

FIG. 2 illustrates a blood pH when the epileptic model rat (GRY rat) is placed in gas containing carbon dioxide or oxygen at each concentration. (Example 1)

FIG. 3 is an explanatory view illustrating a spike index in the present invention. (Example 1)

FIG. 4 illustrates a change in electroencephalograms (spikes) of the epileptic model rat (GRY rat) according to a change in a concentration of mixed gas (carbon dioxide) of inhaled air. (Example 1)

FIG. 5 illustrates results of a spike index of electroencephalograms (spikes) of the epileptic model rat (GRY rat) according to a change in a concentration of mixed gas (carbon dioxide) of inhaled air. (Example 1)

FIG. 6 illustrates a blood concentration of carbon dioxide when the epileptic model rat (GRY rat) is placed in mixed gas of inhaled air containing carbon dioxide or oxygen at each concentration. (Example 2)

FIG. 7 illustrates a blood concentration of oxygen when the epileptic model rat (GRY rat) is placed in mixed gas of inhaled air containing carbon dioxide or oxygen at each concentration. (Example 2)

FIG. 8 illustrates a blood concentration of a bicarbonate ion when the epileptic model rat (GRY rat) is placed in mixed gas of inhaled air containing carbon dioxide or oxygen at each concentration. (Example 2)

FIG. 9 illustrates measurement results of a blood pH, a blood concentration of carbon dioxide, a blood concentration of oxygen, and a blood concentration of a bicarbonate ion in administration (15 minutes to 60 minutes) of 10% (v/v) carbon dioxide or 17% (v/v) oxygen. (Example 3)

FIG. 10 illustrates an electroencephalogram for five minutes before the administration of 10% carbon dioxide and an electroencephalogram for five minutes from 25 seconds after starting the administration. (Example 4)

FIG. 11 illustrates results of a spike index every 15 minutes before and after the administration of 10% carbon dioxide. (Example 4)

FIG. 12 illustrates a blood concentration of acetazolamide and a blood pH when acetazolamide is administered to the epileptic model rat (GRY rat). (Comparative Example 1)

FIG. 13 illustrates a change in electroencephalograms (spikes) of the epileptic model rat (GRY rat) according to administration of acetazolamide. (Comparative Example 1)

FIG. 14 illustrates a blood pH of an epileptic model rat (Kyo811 rat) before and after a seizure of febrile seizures, and after a treatment with mixed gas of inhaled air (carbon dioxide) (Reference Example 1)

FIG. 15 illustrates a change in electroencephalograms (spikes) of the epileptic model rat (Kyo811 rat) according to a change in a concentration of mixed gas of inhaled air (carbon dioxide). (Reference Example 1)

FIG. 16 illustrates results of a seizure duration (second) of the epileptic model rat (Kyo811 rat) according to a change in a concentration of mixed gas of inhaled air (carbon dioxide). (Reference Example 1)

FIG. 17 is a cross-sectional view illustrating a first embodiment of an inhalation gas device for a therapy of a disease of the invention;

FIG. 18 is a cross-sectional view illustrating a second embodiment of an inhalation gas device for a therapy of a disease of the invention; and

FIG. 19 is a cross-sectional view illustrating a third embodiment of an inhalation gas device for a therapy of a disease of the invention.

### DESCRIPTION OF EMBODIMENTS

The present invention relates to a therapeutic agent for a disease accompanied by epileptiform discharges, characterized by containing carbon dioxide as an active ingredient. In the present invention, the disease accompanied by epileptiform discharges is not limited to epilepsy, but means any disease accompanied by epileptiform discharges. Here, the epileptiform discharge means spike, spike and wave, spike-and-wave complex, sharp wave, sharp and slow waves, or the like. In the present invention, specific examples of the disease accompanied by epileptiform discharges include epilepsy, cerebrovascular disorders, and metabolic abnormalities (hypoglycemia, electrolyte abnormalities). Febrile seizures not relating to epilepsy are not included in the scope of the present invention. In the present invention, the disease accompanied by epileptiform discharges is preferably epilepsy. A difference between epilepsy and febrile seizures is as follows. That is, febrile seizure is a benign disease which occurs when a person has fever. Epilepsy is developed when a person has no fever, and a symptom, duration, and prognosis tend to become severe.

Higher organisms including humans produce carbon dioxide by consuming oxygen in the body in order to obtain energy necessary for life activities. Carbon dioxide generated in the body is transferred to the lung through venous blood. The lung releases carbon dioxide from the blood, and incorporates oxygen into the blood by breathing. Various organic acids are synthesized in the body by life activities. By an action of regulation mechanisms, the pH normal value is maintained at 7.35 to 7.45 as an environment in the body.

The therapeutic agent of the present invention for a disease accompanied by epileptiform discharges contains carbon dioxide as an active ingredient. In the present invention, carbon dioxide is used for temporarily lowering a blood pH to a value lower than 7.4 and not interfering with life. Specifically, the blood pH is only required to be temporarily lowered to 7.25-7.40, preferably to 7.30-7.35. In order to lower the blood pH as described above, carbon dioxide gas can be inhaled to be used. Specifically, the therapeutic agent of the present invention for a disease of the present invention can be a therapeutic agent for inhalation, containing carbon dioxide as an active ingredient. The present invention further relates to a medical gas bottle or a medical inhalation gas device for a therapy of a disease accompanied by epileptiform discharges. The therapeutic agent for a disease accompanied by epileptiform discharges, containing carbon dioxide as an active ingredient, may be filled in a medical gas bottle, or the medical gas bottle may be connected to a medical inhalation gas device. Carbon dioxide is filled in such a therapeutic agent for inhalation, medical gas bottle, or medical inhalation gas device such that the concentration of carbon dioxide in inhaled gas upon inhalation is from 1 to 10% (v/v), preferably from 3 to 10% (v/v), and more preferably from 5 to 10% (v/v). It is said that the following usually occurs according to the concentration of carbon dioxide gas in the air. That is, a concentration of 1 to 2% (v/v) causes discomfort. A concentration of 3 to 4% (v/v) stimulates the respiratory center, and causes such a symptom as increase in respiration, elevation in pulse rate, headache, or dizziness. A concentration of 6 to 7% (v/v) makes breathing difficult. A concentration of 7 to 10% (v/v) causes unconsciousness in a few minutes. Therefore, the concentration of carbon dioxide used in the present invention is only required to be able to temporarily lower the blood pH and not to interfere with life upon inhalation.

Carbon dioxide is a colorless and odorless gas at a normal temperature and pressure, and is sublimated at −79° C. to become a solid (dry ice). Carbon dioxide is soluble in water relatively well, and an aqueous solution thereof (carbonic acid) is weakly acidic. Furthermore, an aqueous solution and a solid of a hydroxide of an alkali metal and an alkaline earth metal absorb carbon dioxide, and generate a carbonate or a bicarbonate. Carbon dioxide is liquefied under temperature and pressure conditions of the triple point (−56.6° C., 0.52 MPa) or more. Carbon dioxide becomes a supercritical state under temperature and pressure conditions exceeding the critical point (31.1° C., 7.4 MPa), and has features of both gas and liquid. Carbon dioxide in these states is referred to as compressed carbon dioxide or high-density carbon dioxide. Carbon dioxide contained in the therapeutic agent for inhalation, the medical gas bottle, or the medical inhalation gas device of the present invention is only required to be gas upon inhalation. A generation source of carbon dioxide gas may be gas, liquid, or solid such as dry ice. The therapeutic agent for a disease accompanied by epileptiform discharges of the present invention can contain components of the normal air (nitrogen, oxygen, argon) in addition to carbon dioxide. The therapeutic agent may further contain a gas component which does not adversely affect a living body, for example, helium.

The present invention relates to the above-described therapeutic agent for a disease accompanied by epileptiform discharges, gas bottle for a therapy of a disease, and inhalation gas device for a therapy of a disease, and also relates to a method for treating a disease accompanied by epileptiform discharges, characterized in that the therapeutic agent for a disease is inhaled using a gas bottle for a therapy of a disease accompanied by epileptiform discharges or an inhalation gas device for a therapy of a disease accompanied by epileptiform discharges. For example, by placing the gas bottle or device of the present invention for a therapy of a disease accompanied by epileptiform discharges at homes or educational institutions or by carrying a small gas bottle, when a disease accompanied by epileptiform discharges occur, it is possible to easily reduce a symptom such as a seizure accompanied by the disease by inhaling, from the bottle, a therapeutic agent containing carbon dioxide as an active ingredient, specifically carbon dioxide gas, the concentration of which is from 1 to 10% (v/v), preferably from 3 to 10% (v/v), and more preferably from 5 to 10% (v/v) in inhaled gas.

### First Example of Inhalation Gas Device for Therapy of Disease

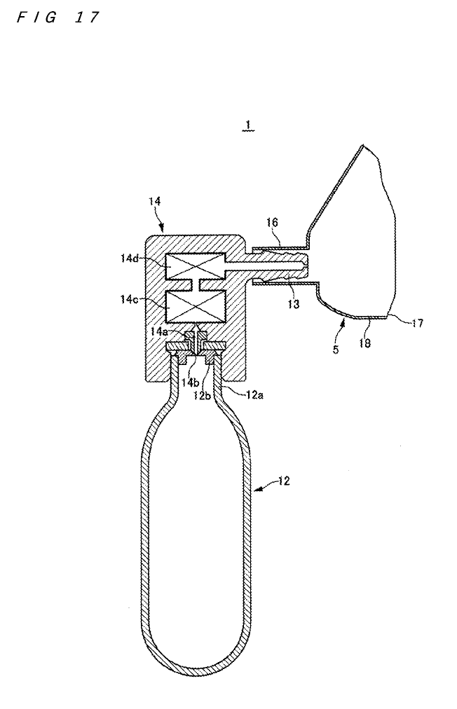

FIG. 17 illustrates a first embodiment of an inhalation gas device 1 for a therapy of a disease of the invention. The inhalation gas device for a therapy of a disease illustrated in this embodiment includes a portable medical gas bottle 12 in which the above-mentioned therapeutic agent for a disease accompanied by epileptiform discharges is compressed and filled, a gas extraction member 14 which is attached to an opening of the medical gas bottle 12 to discharge the therapeutic agent for a disease in the medical gas bottle 12 from a discharge nozzle 13, and a gas inhalation mask 5 connected to the discharge nozzle 13 of the gas extraction member 14. The gas extraction member 14 and the gas inhalation mask 5 constitute a medical inhalation gas device.

The medical gas bottle 12 is a compact gas container excellent in portability with an internal volume of about 10 to 100 ml, and a therapeutic agent for a disease accompanied by epileptiform discharges containing carbon dioxide as an active ingredient as an inhalation gas is compressed and filled inside the medical gas bottle 12. Further, the opening of a container neck portion 12a of the medical gas bottle 12 before the use is sealed with a sealing plate 12b, and a male screw portion is provided on the outer periphery of the container neck portion 12a.

The gas extraction member 14 is provided with a female screw portion screwed to the male screw portion of the container neck portion 12a on the lower inner periphery, and has a needle 14b for causing the interior of the medical gas bottle 12 to communicate with an internal flow path 14a of the gas extraction member 14, by perforating the sealing plate 12b when the gas extraction member 14 is screwed into the container neck portion 12a, a depressurizing portion 14c, a flow rate adjusting portion 14d, and the discharge nozzle 13 which discharges the inhalation gas depressurized and subjected to flow-rate controlling.

The gas inhalation mask 5 can be made of a hard resin material, for example, polycarbonate, vinyl chloride, or the like which does not permeate the gas and is enough to be able to maintain the shapes of an inhalation shape portion 17 and an enlarged portion 18 to be described later. A tubular base side connecting portion 16 connected to the discharge nozzle 13 is provided on the mask base portion side of the gas inhalation mask 5. By fitting and connecting the discharge nozzle 13 to the base side connecting portion 16, the gas inhalation mask 5 is in a state of being attached to the medical gas bottle 12. Also, on the mask distal end side of the gas inhalation mask 5, an inhalation shape portion 17 in which a distal end edge covers the periphery of the mouth and nose of the inhaler and abuts on the face is provided, and the inhalation shape portion 17 and the base side connecting portion 16 are connected to each other by a conical enlarged portion 18 which gradually expands in diameter toward the inhalation shape portion 17.

In the inhalation gas device for a therapy of a disease according to the invention, since an inhaler inhales a therapeutic agent for a disease accompanied by epileptiform discharges via the gas extraction member 14 and the gas inhalation mask 5, it is possible to effectively perform the inhalation and administration of the therapeutic agent for a disease.

### Second Example of Inhalation Gas Device for Therapy of Disease

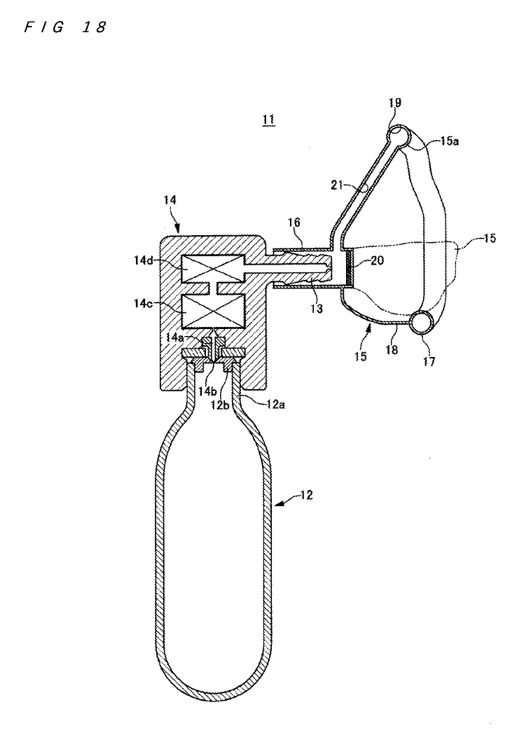

FIG. 18 illustrates a second embodiment of the inhalation gas device for a therapy of a disease of the invention. Further, in the following description, the same constituent elements as those of the inhalation gas device for a therapy of a disease illustrated in the first embodiment are denoted by the same reference numerals, and a detailed description thereof will not be provided.

The gas inhalation mask 15 used in the inhalation gas device 11 for a therapy of a disease illustrated in this embodiment is formed entirely of a material having flexibility, for example, a material through which a gas is hard to pass, such as polyvinyl chloride, silicone, and latex. Further, a peripheral edge gas introducing portion 19 having a small-diameter hollow shape is provided on the peripheral edge of the distal end side opening portion 15a of the gas inhalation mask 15. The peripheral edge gas introducing portion 19 expands due to the introduction of the therapeutic agent for a disease discharged from the discharge nozzle 13 to become a shape of the peripheral edge portion of the inhalation shape portion 17. The peripheral edge gas introducing portion 19 is formed to be contractible and foldable state when the therapeutic agent for a disease is not introduced, and is formed to be brought into a state of being folded or wrapped around the outer periphery of the medical gas bottle 12 before the use of the inhalation gas device 11 for a therapy of a disease.

A resistance member 20 that gives resistance against the flow of the therapeutic agent for a disease discharged from the discharge nozzle 13 and flowing from the interior of the enlarged portion 18 toward the inhalation shape portion 17 is provided on the distal end side of the base side connecting portion 16. As the resistance member 20, various materials and articles can be used as long as a proper gas flow resistance can be obtained. For example, a sintered filter, an orifice, a valve, or the like can be used. Further, a gas guide flow path 21, which introduces the therapeutic agent for a disease discharged from the discharge nozzle 13 into the peripheral edge gas introducing portion 19, branches from a space between the resistance member 20 and the discharge nozzle 13 in the base side connecting portion 16. The gas guide flow path 21 can be formed by another pipe material or the like, but by integrally formed with the enlarged portion 18, the manufacturing cost of the gas inhalation mask 15 can be reduced.

The resistance member 20 gives resistance against the flow of the therapeutic agent for a disease discharged from the discharge nozzle 13 and directed toward the inhalation shape portion 17, thereby preferentially introduces the therapeutic agent for a disease discharged from the discharge nozzle 13 into the peripheral edge gas introducing portion 19 from the gas guide flow path 21, and expands the peripheral edge gas introducing portion 19 with the therapeutic agent for a disease to make the mask distal end side have a shape of the inhalation shape portion 17, before an inhaler starts the inhalation of the therapeutic agent for a disease from the inhalation shape portion 17. Further, after the gas guide flow path 21 and the peripheral edge gas introducing portion 19 are filled by the introduction of the therapeutic agent for a disease and the pressure rises, a predetermined amount of the therapeutic agent for a disease flows from the base side connecting portion 16 toward the distal end side opening portion 15a through the resistance member 20, and is supplied to the mouth and nose of the inhaler from the inhalation shape portion 17.

The inhalation gas device 11 for a therapy of a disease thus formed can be carried in a state in which the gas inhalation mask 15 is attached to the medical gas bottle 12. In the state before the use, since the therapeutic agent for a disease is not introduced into the peripheral edge gas introducing portion 19 and the peripheral edge gas introducing portion 19 is in a contracted state, as illustrated by an imaginary line in FIG. 18, the gas inhalation mask 15 is in an expanded state or in an appropriately folded small state, and can be stored in a small pouch, a bag, or the like together with the medical gas bottle 12, and the contracted gas inhalation mask 15 does not become an obstacle.

Further, when the inhalation of the therapeutic agent for a disease becomes necessary due to an occurrence of a disease accompanied by epileptiform discharges, the inhalation gas device 11 for a therapy of a disease is extracted from a pouch, a bag, or the like, and the gas extraction member 14 is screwed into the medical gas bottle 12. As a result, the sealing plate 12b is perforated with a needle 14b, and the therapeutic agent for a disease in the medical gas bottle 12 is discharged from the discharge nozzle 13 through the internal flow path 14a, the depressurizing portion 14c, and the flow rate adjusting portion 14d.

Substantially all amount of the therapeutic agent for a disease discharged from the discharge nozzle 13 flows into the peripheral edge gas introducing portion 19 through the gas guide flow path 21 by the action of the resistance member 20, and expands the peripheral edge gas introducing portion 19 to make the distal end edge of the distal end side opening portion 15a a predetermined inhalation shape portion 17. After the peripheral edge gas introducing portion 19 expands, substantially all amount of the therapeutic agent for a disease passes through the resistance member 20 and is supplied into the gas inhalation mask 15 having a predetermined shape covering the periphery of the mouth and nose of the inhaler, and the inhaler is in a state of inhaling the therapeutic agent for a disease.

Therefore, before and after the use, the inhalation gas device 11 for a therapy of a disease having the gas inhalation mask 15 attached thereto can be compactly stored in a pouch, a bag, or the like, and the gas inhalation mask 15 has a predetermined shape for instantly covering the periphery of the mouth and the nose of the inhaler at the time of use. Thus, it is possible to effectively inhale and administer the therapeutic agent for a disease in the medical gas bottle 12 and the therapeutic agent for a disease does not become wasteful.

### Third Example of Inhalation Gas Device for Therapy of Disease

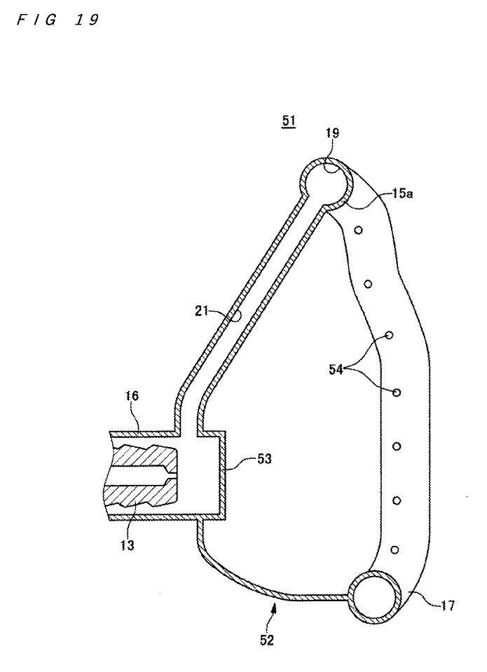

FIG. 19 illustrates a third embodiment of an inhalation gas device for a therapy of a disease of the invention. In the following description, the same constituent elements as those of the inhalation gas device for a therapy of a disease illustrated in the first embodiment and the second embodiment are denoted by the same reference numerals, and a detailed description thereof will not be provided. In particular, since the medical gas bottle 12 and the gas extraction member 14 can adopt the same configuration as those of the first embodiment and the second embodiment, they are not illustrated also.

Similarly to the gas inhalation mask 15 illustrated in the second embodiment, a gas inhalation mask 52 used in an inhalation gas device 51 for a therapy of a disease illustrated in the present embodiment is entirely formed of a material having flexibility. At a distal end portion of the base side connecting portion 16 provided on the mask base portion side, a shielding member 53 for shielding the flow of the therapeutic agent for a disease from the discharge nozzle 13 toward the inhalation shape portion 17 is provided, instead of the resistance member 20. Also, on the inner periphery of the peripheral edge gas introducing portion 19, a plurality of inhalation gas discharge holes 54 which discharges the therapeutic agent for a disease, which is introduced from the gas guide flow path 21 into the peripheral edge gas introducing portion 19, toward the inside of the enlarged portion 18 is provided.

Therefore, when the gas extraction member is screwed into the medical gas bottle to start the supply of the therapeutic agent for a disease from the discharge nozzle 13 in the gas inhalation mask 52 illustrated in this embodiment, all amount of the therapeutic agent for a disease is in a state of being introduced from the gas guide flow path 21 to the peripheral edge gas introducing portion 19 by the action of the shielding member 53, being injected from the inhalation gas discharge holes 54 into the gas inhalation mask 15, and being supplied from the inhalation shape portion 17 to the mouth and nose of the inhaler.

An inner diameter caliber and the number of installations of the inhalation gas discharge holes 54 may be set such that the peripheral edge gas introducing portion 19 can be sufficiently expanded with the therapeutic agent for a disease flowing in from the gas guide flow path 21, and the amount of the therapeutic agent for a disease injected into the gas inhalation mask 15 becomes an amount of gas required by the inhaler. By adjusting the inner diameter of the gas guide flow path 21 or by adjusting the number of the gas guide flow paths 21, while introducing the therapeutic agent for a disease having an amount and pressure enough for expansion into the peripheral edge gas introducing portion 19, the amount of the therapeutic agent for a disease to be supplied into the mask and inhaled by the inhaler can be adjusted to an optimum amount.

Further, in the gas inhalation mask 52 illustrated in the third embodiment, the inhalation gas discharge hole 54 is provided in the peripheral edge gas introducing portion 19 to supply the therapeutic agent for a disease into the gas inhalation mask 15. However, the inhalation gas discharge hole may be provided in the shielding member of the base side connecting portion 16 to supply a part of the therapeutic agent for a disease into the gas inhalation mask 15, and the inhalation gas discharge hole may be provided in the gas guide flow path 21 to supply a part of the therapeutic agent for a disease into the gas inhalation mask 15.

Further, a gas permeable member, for example, a nonwoven fabric, a film, a mesh material, a porous body, or the like can also be used as an appropriate shape in place of the inhalation gas discharge hole, the whole of the portion becoming the inside of the gas inhalation mask 15 can also be made gas permeable, and the entire gas inhalation mask 15 including the peripheral edge gas introducing portion 19 and the gas guide flow path can also be formed in a hollow shape.

The base side connecting portion of the gas inhalation mask can be formed to have a structure corresponding to the shape of the discharge nozzle provided in the gas extraction member. In addition, the peripheral edge gas introducing portion in the gas inhalation mask can be provided over the entire circumference in a ring shape. However, as long as the shape of the inhalation shape portion can be maintained at the time of expansion, it is not necessary to form the peripheral edge gas introducing portion in a ring shape, and the peripheral edge gas introducing portion may be divided into a plurality sections to provide the gas guide flow path in each of them. Further, it is also possible to provide a flow rate adjusting mechanism on the gas extraction member. An on-off valve may be provided in place of the sealing plate of the medical gas bottle and the needle of the gas extraction member. It is also possible to connect the gas inhalation mask to the discharge nozzle of the gas extraction member via the extension tube.

### REFERENCE SIGNS LIST

1 INHALATION GAS DEVICE FOR THERAPY OF DISEASE
5 GAS INHALATION MASK
11 INHALATION GAS DEVICE FOR THERAPY OF DISEASE
12 MEDICAL GAS BOTTLE
12a CONTAINER NECK PORTION
12b SEALING PLATE
13 DISCHARGE NOZZLE
14 GAS EXTRACTION MEMBER
14a INTERNAL FLOW PATH
14b NEEDLE
14c DEPRESSURIZING PORTION
14d FLOW RATE ADJUSTING PORTION
15 GAS INHALATION MASK
15a DISTAL END SIDE OPENING PORTION
16 BASE SIDE CONNECTING PORTION
17 INHALATION SHAPE PORTION
18 ENLARGED PORTION
19 PERIPHERAL EDGE GAS INTRODUCING PORTION
20 RESISTANCE MEMBER
21 GAS GUIDE FLOW PATH
51 INHALATION GAS DEVICE FOR THERAPY OF DISEASE
52 GAS INHALATION MASK
53 SHIELDING MEMBER
54 INHALATION GAS DISCHARGE HOLE

### EXAMPLES

The present invention will be described specifically by showing Examples, Comparative Examples, and Reference Examples, in order to assist in understanding the present invention. However, of course, the present invention is not limited thereto.

### (Example 1) Administration of Carbon Dioxide to Epileptic Model Rats

In the present Example, carbon dioxide at each concentration was administered to GRY rats. The blood pH, the blood concentration of carbon dioxide, and electroencephalograms were measured. A simultaneous video-electroencephalographic recording technology was established, and a spike index as an index of evaluation of a seizure was measured.

1) Material and Method

[Epileptic Model Rat]

In the present Example, a GRY rat (groggy rat, Cacnala) was used as an epileptic model rat. A GRY rat constantly develops an epileptic seizure in normal conditions, and therefore is an extremely excellent system to evaluate a seizure by subjecting the GRY rat to a treatment. The GRY rat has a mutation in an al subunit of a P/Q-type voltage-dependent calcium channel Cav2.1 which encodes Cacnala gene, and is an epileptic model rat which mainly develops ataxia and absence seizure. The GRY rat takes a mode of autosomal recessive inheritance. The 752nd nucleotide “T” of the Cacnala gene has mutated to “A”. As a result, a codon “ATG” to specify methionine which is the 251st amino acid has mutated to a codon “AAG” to specify lysine (M251K). It has been reported that this mutation is located at extracellular p-loop forming a pore of a calcium channel domain 1 and that this mutation (M251K) results in abnormality of a function of the voltage-dependent calcium ion channel Cav2.1 of the rat (Reference Literatures 1 and 2). The GRY rats with this mutation show extension abnormalities of hind limbs, walking abnormalities, and ataxia, and a seizure which is accompanied with spike and wave discharges of 7-8 Hz on the electroencephalograms from 6-8 weeks of age. The present model rats were dispensed from Institute of Laboratory Animals, Graduate School of Medicine, Kyoto University.

[Surgery of Embedding Electrode for Measurement of Electroencephalograms]

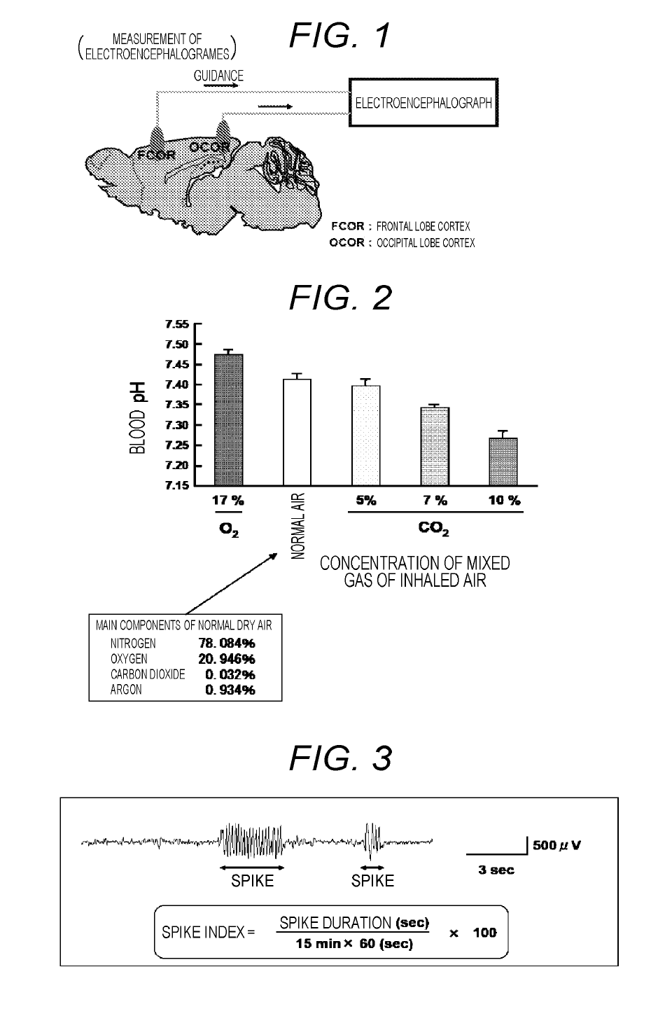

Male GRY rats were anesthetized by administering 35 mg/kg of pentobarbital sodium (Nembutal, Sumitomo Dainippon Pharma Co. Ltd.) intraperitoneally, and were fixed to a brain stereotaxic fixing apparatus (SR-5M, NARISHIGE Group). The head was shaved, the skin was incised along the median line, and the skull was exposed. A small hole was opened on the frontal lobe cortex and the occipital lobe cortex on both sides using a dental drill (FALCON, Morita Corporation). A stainless steel screw electrode (FUKUOKA RASHI) having a diameter of 0.9 mm and a length of 1.7 mm and connected a stainless steel wire having a diameter of 0.2 mm (M T Giken) was embedded in the hole (refer to FIG. 1). A screw electrode was embedded in the root of nose as a Z electrode, and a screw electrode was embedded in the cerebellum as an indifferent electrode. Each electrode was connected to a connector socket (Hirose Electric Co. Ltd.), and fixed to the skull with dental cement (Uni Fast II, GC Dental Products).

[Control of Concentration of Carbon Dioxide]

In order to control a concentration of carbon dioxide, multi-gas incubator (MCO-5M; Sanyo Electric Co., Ltd.) was used as a multi-gas concentration control apparatus to adjust the concentration of carbon dioxide in the incubator. A concentration of oxygen was also adjusted for comparison. Main components of the normal dry air are 0.032% (v/v) of carbon dioxide, 20.946% (v/v) of oxygen, and 78.084% (v/v) of nitrogen. Therefore, using the above-described multi-gas concentration control apparatus, the concentration of carbon dioxide was set to 5% (v/v), 7% (v/v), or 10% (v/v), or the concentration of oxygen was set to 17% (v/v). When the concentration of carbon dioxide was adjusted, carbon dioxide gas supplied from a carbon dioxide gas bottle connected to the apparatus was diluted with the air outside. Therefore, carbon dioxide=10% (v/v), oxygen=about 18.85% (v/v), and nitrogen=about 70.28% (v/v). When the concentration of oxygen was set to 17% (v/v), similarly, the concentration was adjusted using nitrogen gas supplied from a nitrogen gas bottle connected to the apparatus. Therefore, carbon dioxide=0.026% (v/v), oxygen=about 17% (v/v), and nitrogen=about 82.974% (v/v). It was confirmed using a gas analyzer AGA-2008 (Astec Co. Ltd.) that each concentration in the incubator was correct under the set conditions.

2) Administration of Carbon Dioxide at Each Concentration to Rats

The surgery of embedding an electrode for measurement of electroencephalograms was performed to eight male GRY rats (9 weeks old). After a one-week recovery period, the GRY rats were placed for one hour in the multi-gas concentration control apparatus adjusted to a concentration of carbon dioxide of 5% (v/v), 7% (v/v), or 10% (v/v), or a concentration of oxygen of 17% (v/v).

3) Blood pH, Concentration of Carbon Dioxide

Blood samples were collected from the tail vein of the rats placed in the multi-gas concentration control apparatus, and the blood pH and the blood concentration of carbon dioxide were measured using an i-STAT analyzer (Fuso Pharmaceutical Industries, Ltd.) as a blood gas analyzer.

It was confirmed that the blood concentration of carbon dioxide of the rats was increased in accordance with increase in the concentration of carbon dioxide in the multi-gas concentration control apparatus to 5% (v/v), 7% (v/v), or 10% (v/v), and that the blood concentration of carbon dioxide of rats was reduced when the concentration of oxygen in the multi-gas concentration control apparatus was changed to 17% (v/v). The blood pH was measured in similar conditions. As a result, the blood pH was reduced in accordance with increase in the concentration of carbon dioxide in the multi-gas concentration control apparatus to 5% (v/v), 7% (v/v), or 10% (v/v), and that the blood pH was increased when the concentration of oxygen in the multi-gas concentration control apparatus was changed to 17% (v/v). This makes it clear that the blood concentration of carbon dioxide was increased and the blood pH was reduced when the concentration of carbon dioxide in the multi-gas concentration control apparatus was changed to 5% (v/v), 7% (v/v), or 10% (v/v) (FIG. 2).

When the concentration of carbon dioxide is increased, a high concentration of carbon dioxide is taken in as inhaled air. As a result, respiratory acidosis occurs. On the other hand, when the concentration of oxygen is reduced, a respiratory rate is increased. As a result, a large amount of carbon dioxide is released, and respiratory alkalosis occurs. Here, the acidosis means a state in which an original acid-base equilibrium of blood is kept to have a pH of 7.4 but the equilibrium goes to an acidic side. The alkalosis means a state in which the acid-base equilibrium of blood goes to a basic side.

The pH of blood or body fluids is represented by the following Henderson-Hasselbalch equation.

pH=pKa+log [HCO₃⁻]/[CO₂] (pKa=6.1)

A bicarbonic acid-carbon dioxide (HCO₃⁻/CO₂) buffer system is the most important pH buffer system of blood or body fluids. Carbon dioxide reacts with a water molecule in the body to generate bicarbonic acid.

CO₂+H₂O←→HCO₃⁻+H⁺

[CO₂] is regulated by breathing, and [HCO₃] is regulated by the liver and kidney. Therefore, it is considered that the therapeutic agent of the present invention for a disease accompanied by epileptiform discharges causes respiratory acidosis by increasing the concentration of carbon dioxide of inhaled air with carbon dioxide contained as an active ingredient, and suppresses epileptiform discharges.

In a case of a healthy person, the blood pH is usually 7.4 (normal range of female=pH 7.40±0.015, normal range of male=pH 7.39±0.015). It is said that the blood pH of 7.0 or less or 7.8 or more makes life support impossible. In the present Example, the pH was 7.27 in an environment of 10% (v/v) carbon dioxide.

4) Evaluation of Seizure

The rats placed in the multi-gas concentration control apparatus adjusted to a concentration of carbon dioxide of 5% (v/v), 7% (v/v), or 10% (v/v) were subjected to simultaneous video-electroencephalographic recording. Rats placed in the air were used as a control.

Electroencephalograms of the frontal lobe cortex and the occipital lobe cortex of the rats were measured using an electroencephalograph (Neurofax EEG-1200, NIHON KOHDEN CORPORATION). A spike index was used as an index of evaluation of a seizure. The spike index represents a ratio of duration of a spike in measurement time (15 minutes). The spike means a waveform indicating a sharp change in potential in a short time (refer to FIG. 3).

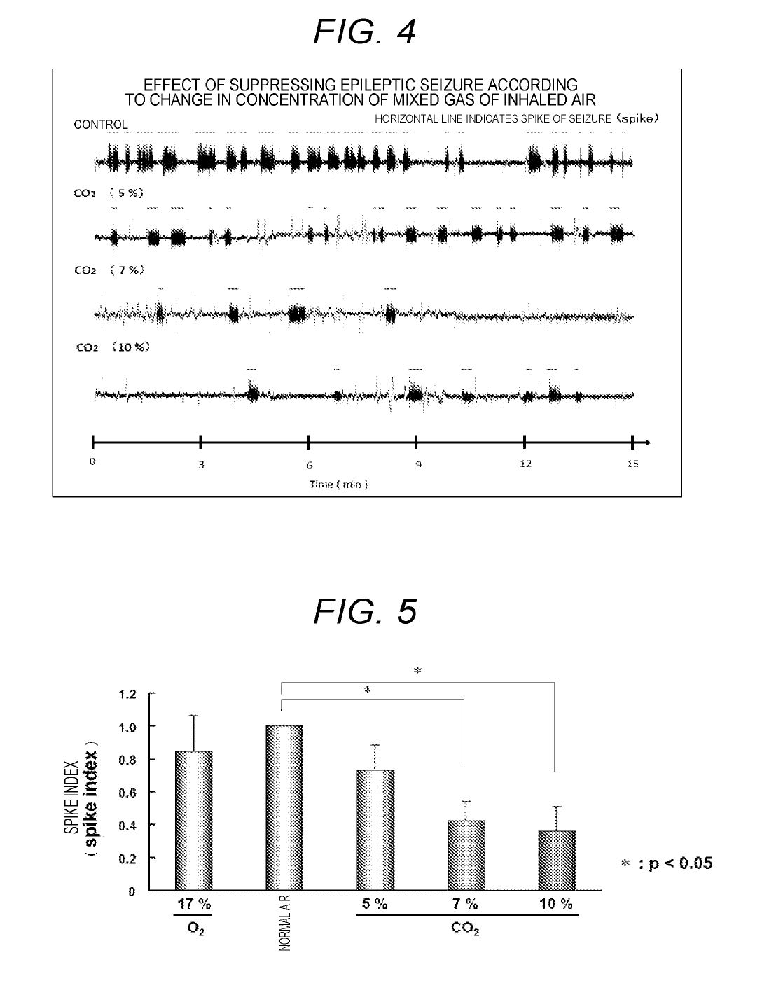

FIG. 4 illustrates electroencephalograms according to a change in the concentration of mixed gas of inhaled air. FIG. 5 illustrates results of the spike index. As a result, it was confirmed that epileptiform discharges, that is, duration of a spike could be suppressed significantly by changing the concentration of carbon dioxide to 7% (v/v) or 10% (v/v).

### (Example 2) Administration of Carbon Dioxide to Epileptic Model Rats 2

Male GRY rats (9 weeks old) were placed for one hour in the multi-gas concentration control apparatus adjusted to a concentration of carbon dioxide of 5% (v/v), 7% (v/v), or 10% (v/v), or a concentration of oxygen of 17% (v/v) by the same manner as in Example 1 (six rats for each).

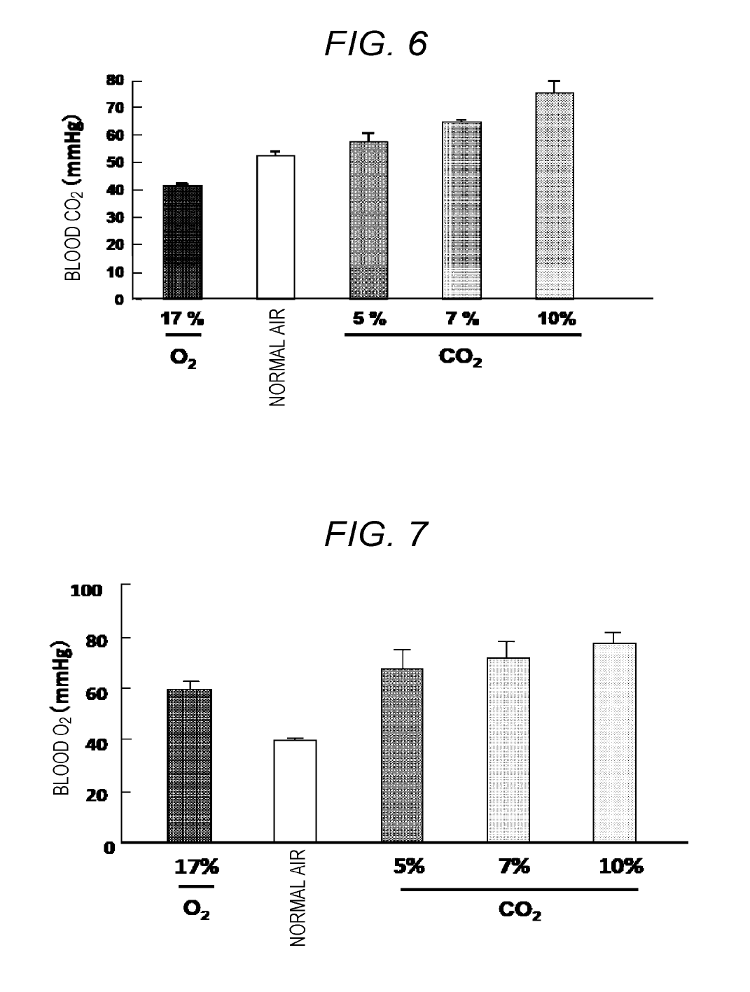

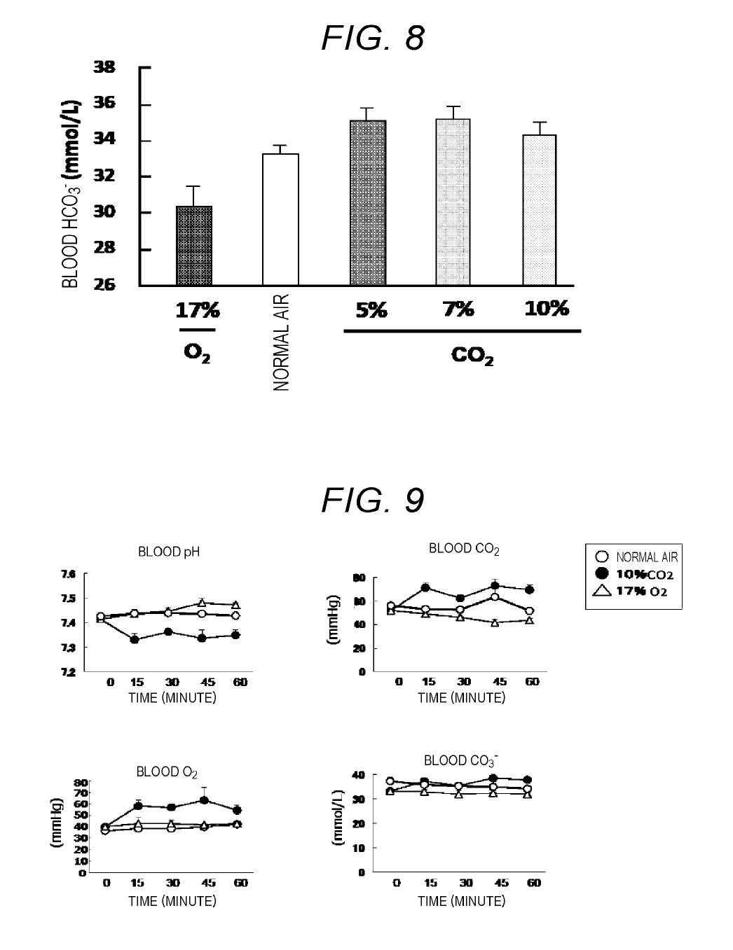

The blood concentration of carbon dioxide, the blood concentration of oxygen, and the blood concentration of a bicarbonate ion were measured for model rats under each condition. It was confirmed that the blood concentration of carbon dioxide of the rats was increased in accordance with increase in the concentration of carbon dioxide to 5% (v/v), 7% (v/v), or 10% (v/v), and that the blood concentration of carbon dioxide of rats was reduced when the concentration of oxygen was changed to 17% (v/v) (FIG. 6). FIGS. 7 and 8 illustrate the blood concentration of oxygen and the blood concentration of a bicarbonate ion under similar conditions.

The results in Examples 1 and 2 make it clear that the blood concentration of carbon dioxide was increased and the blood pH was reduced when the concentration of carbon dioxide in the multi-gas concentration control apparatus was changed to 5% (v/v), 7% (v/v), or 10% (v/v).

### (Example 3) Administration of Carbon Dioxide to Epileptic Model Rats 3

In the present Example, when 10% (v/v) carbon dioxide or 17% (v/v) oxygen was administered to GRY rats in different administration times, the blood pH, the blood concentration of carbon dioxide, the blood concentration of oxygen, and the blood concentration of a bicarbonate ion were measured.

1) Material and Method

A model animal was the same as in Example 1, and control of a concentration of carbon dioxide was performed by the same manner as in Example 1. Carbon dioxide or oxygen was administered to rats by placing the rats in the multi-gas concentration control apparatus adjusted to a concentration of carbon dioxide of 10% (v/v) or a concentration of oxygen of 17% (v/v) for 15 minutes, 30 minutes, 45 minutes, or 60 minutes (5-6 rats for each). Rats placed in the multi-gas concentration control apparatus adjusted to a concentration of the normal air were used as a control. The blood pH and concentrations of carbon dioxide, oxygen, and a bicarbonate ion were measured by the same manner as in Example 1 or 2.

2) Measurement Results of Blood pH and Concentrations of Carbon Dioxide, Oxygen, and Bicarbonate Ion

Measurement results of the blood pH, the blood concentration of carbon dioxide, the blood concentration of oxygen, and the blood concentration of a bicarbonate ion in the above-described rats are illustrated in FIG. 9. It was confirmed that administration of 10% (v/v) carbon dioxide increased the blood concentration of carbon dioxide and reduced the blood pH for 15 to 60 minutes.

### (Example 4) Administration of Carbon Dioxide to Epileptic Model Rats 4

In the present Example, GRY rats were administered with 10% (v/v) carbon dioxide, and subjected to simultaneous video-electroencephalographic recording, and an effect of suppressing an epileptic seizure (spike index) was measured.

1) Material and Method

A model animal was the same as in Example 1, and a surgery of embedding an electrode for measurement of electroencephalograms, control of a concentration of carbon dioxide, and measurement of electroencephalograms were performed by the same manner as in Example 1. Carbon dioxide was administered to rats by placing the rats in the multi-gas concentration control apparatus adjusted to a concentration of carbon dioxide of 10% (v/v) (9 rats).

2) Evaluation of Seizure

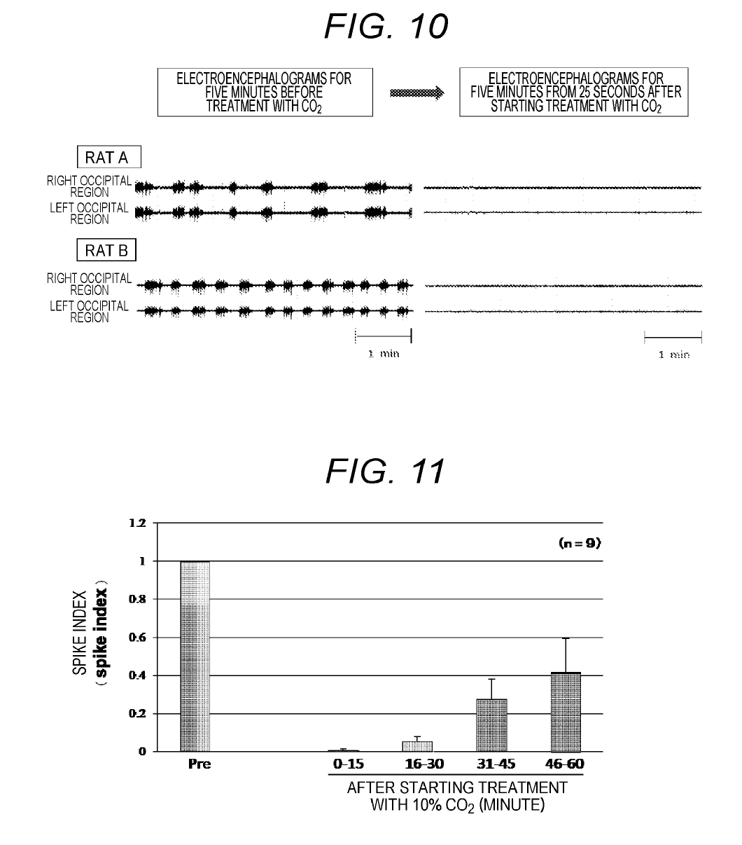

FIG. 10 illustrates results of measuring, by the same manner as in Example 1, electroencephalograms of the frontal lobe cortex and the occipital lobe cortex of the rats to which 10% (v/v) carbon dioxide had been administered by placing the rats in the multi-gas concentration control apparatus. FIG. 10 illustrates an electroencephalogram for five minutes before the administration of 10% (v/v) carbon dioxide and an electroencephalogram for five minutes from 25 seconds after starting the administration. As a result, it was confirmed that epileptiform discharges, that is, duration of a spike could be suppressed significantly by administering the therapeutic agent of the present invention (10% (v/v) carbon dioxide).

3) Experiment of Suppressing Seizure

By placing the rats in the multi-gas concentration control apparatus, the rats were administered with 10% (v/v) carbon dioxide, and subjected to simultaneous video-electroencephalographic recording every 15 minutes for one hour, and an effect of suppressing an epileptic seizure (spike index) was measured before and after the administration of carbon dioxide.

FIG. 11 illustrates results of a spike index every 15 minutes before and after the administration of 10% (v/v) carbon dioxide. When the spike index for 15 minutes before the administration of carbon dioxide was assumed to be 1, an average spike index was 0.0074 in the initial 15 minutes (0-15 minutes), was 0.054 in the subsequent 15 minutes (16-30 minutes), was 0.275 in the subsequent 15 minutes (31-45 minutes), and was 0.416 in the subsequent 15 minutes (46-60 minutes).

From the above results, it was confirmed that the therapeutic agent of the present invention (10% (v/v) carbon dioxide) could suppress a seizure almost completely 15 minutes after the administration, that this effect of suppressing a seizure could last at least until 30 minutes after the administration, and that the suppressing effect gradually decreased. The above Example 3 indicates that the blood concentration of carbon dioxide and the blood pH did not change much for the initial one hour after the administration of carbon dioxide, but that the effect of suppressing a seizure gradually decreased 31-45 minutes and 46-60 minutes after the administration of 10% (v/v) carbon dioxide. It is considered that the effect of suppressing a seizure gradually decreased due to some reasons, in spite of maintaining the decreased level of the blood pH for 31-60 minutes after the administration of carbon dioxide.

### (Comparative Example 1) Comparative Example Using Acetazolamide

Acetazolamide (Acetazolamide, trade name “Diamox®”, “Acetamox®”) is a carbonic anhydrase inhibitor, and inhibits a carbonic anhydrase in a process of producing water and carbon dioxide from carbonic acid. As a result, excretion of sodium bicarbonate is increased and metabolic acidosis is thereby caused.

First, the blood concentration and the blood pH after administration of acetazolamide were measured. Acetazolamide (50 mg/kg) was administered to male GRY rats intraperitoneally. Blood samples were collected from the tail vein before the administration, 15 minute, 30 minute, 45 minutes and 60 minutes after the administration. The blood concentration of acetazolamide, the blood pH, and the blood bicarbonate ion were measured. Saline was administered as a control (8 rats for each). Measurement of the blood concentration of acetazolamide was entrusted to SRL Inc. to be performed. The pH was measured by the same manner as in Example 1.

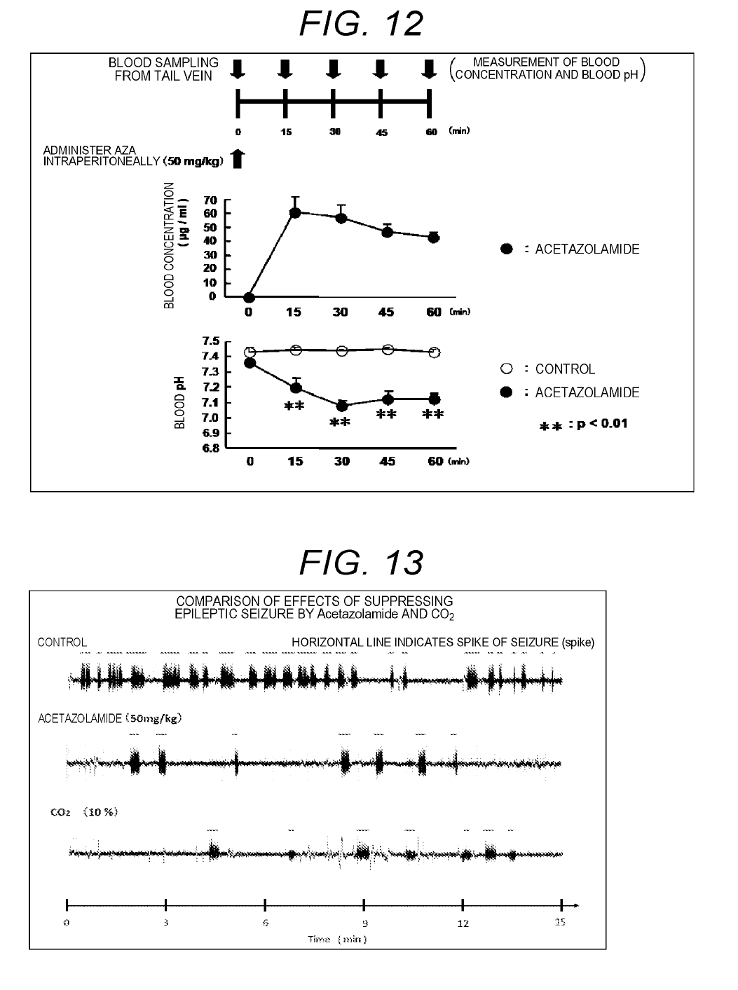

The blood concentration of acetazolamide after the administration of acetazolamide was the highest 15 minutes after the administration and decreased with time (FIG. 12). The blood pH of the acetazolamide administration group was significantly low (on an acidic side) 15 minutes, 30 minute, 45 minutes, and 60 minutes after the administration as compared with the control group (FIG. 12). The blood bicarbonate ion of the acetazolamide administration group was significantly low 15 minutes, 30 minute, 45 minutes, and 60 minutes after the administration as compared with the control group, although data is not illustrated. It was found that the blood pH was acidic within 60 minutes after the administration of acetazolamide in this condition due to non-respiratory (metabolic) acidosis.

Next, electroencephalograms were measured. 9 weeks old GRY rats were fixed to a brain stereotaxic fixing apparatus under anesthesia with pentobarbital, and a chronic electrode for measurement of electroencephalograms was embedded in the frontal lobe cortex and the occipital lobe cortex by the same manner as in Example 1 (refer to FIG. 1). One week after the surgery, acetazolamide (50 mg/kg) was administered intraperitoneally, and the measurement of electroencephalograms was started (administration group: 10 rats, non-administration group: 10 rats). The measurement of electroencephalograms and calculation of the spike index were performed by the same manner as in Example 1. As a result, it has been revealed that acetazolamide significantly suppresses (reduces) the spike duration of a seizure (FIG. 13). Elapsed time of the action showed a correlation with the blood pH and the change in the bicarbonate ion.

As described above, it has been indicated that an epileptic seizure can be suppressed also by the administration of acetazolamide. However, 30 minutes after the administration of acetazolamide (50 mg/kg), the pH was 7.08±0.03. This suggests that the pH may become a very dangerous value for vital (refer to FIG. 12). Acetazolamide is a carbonic anhydrase inhibitor. However, carbon dioxide in the present invention does not act on a specific enzyme activity unlike acetazolamide, and therefore is considered to be safer.

### (Reference Example 1) Administration of Carbon Dioxide to Epileptic Model Rats

In the present Reference Example, 10% (v/v) carbon dioxide was administered to Kyo811 rats. The blood pH and electroencephalograms were measured. The rats were subjected to simultaneous video-electroencephalographic recording, and seizure duration was measured.

1) Material and Method

[Epileptic Model Rat]

In epilepsy, about 80% of patients of intractable Dravet syndrome (formerly, referred to as Severe Myoclonic Epilepsy in Infancy: SMEI), and about 5-10% of patients of benign generalized epilepsy with febrile seizure plus: GEFS+) have missense mutations in a voltage-dependent sodium channel a subunit type 1 (SCN1A) gene. Therefore, it is considered that the mutations in the SCN1A gene are involved in development of febrile seizures (Reference Literatures 3 and 4). In the present Reference Example, using the Kyo811 rats having a mutation in the Scn1a gene as a genetic factor, suppressive effect of seizure by carbon dioxide was examined. This rat is a febrile seizure model rat which induces febrile seizures by a thermal load, and a GEFS+ model rat.

A Kyo811 rat was prepared as a rat with a mutation in the voltage-dependent sodium channel Scn1a gene by the following technology. That is, a mutagen (N-nitro-N-ethylurea; ENU) was introduced into the peritoneal cavity of a male F344 rat, and an artificial mutation was introduced into the DNA of a sperm. Thereafter, the sperm was extracted, and the obtained sperm was subjected to intracytoplasmic sperm injection into an egg of a female rat (Reference Literature 5). As a result of a genetic analysis, in the Kyo811 rat, the 4251th nucleotide “A” of the Scn1a gene has been mutated to “C”, and as result, asparagine (AAT) as the 1417th amino acid has been changed to histidine (CAT) (N1417H). The 1417th asparagine is located in a pore-forming region involved in ion transmission of a sodium ion channel third domain. As a result of a functional analysis of the mutant voltage-dependent sodium channel, it has been found that the N1417H mutant sodium ion channel has an abnormality in the channel function, and easily causes seizure. This mutant homozygous rat causes febrile seizures about 3-4 minutes after the rat is put in a warm bath at 45° C. Therefore, the rat is very useful as a febrile seizure model rat upon a warm bath load. The present model rats were dispensed from Institute of Laboratory Animals, Graduate School of Medicine, Kyoto University.

[Surgery of Embedding Electrode for Measurement of Electroencephalograms]

The surgery was performed by the same manner as in Example 1

[Control of Concentration of Carbon Dioxide]

The control was performed by the same manner as in Example 1.

2) Induction of Febrile Seizures of Rats with Thermal Load

Chronic electrodes for measurement of electroencephalograms were embedded in male Kyo811 rats (5 weeks old) by the same manner as in Example 1 (5 rats for each). After a one-week recovery period, the rats were put into a warm bath at 45° C. for about 3-4 minutes to induce febrile seizures, and electroencephalograms were measured.

3) Administration of Carbon Dioxide to Rats

Immediately after the seizure was induced, the rats were placed in the multi-gas concentration control apparatus adjusted to the concentration of carbon dioxide of 10% (v/v) or the concentration of the normal air, and the situation of the seizure was observed while electroencephalograms were measured continuously.

4) Blood pH

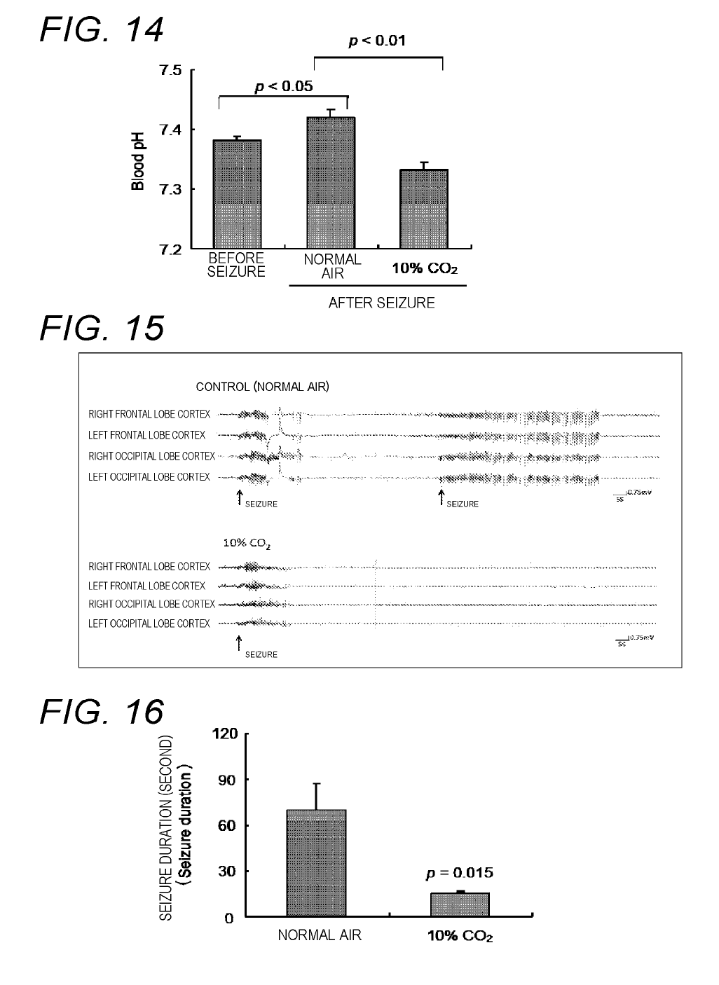

As soon as the seizures of the rats placed in the multi-gas concentration control apparatus were terminated, blood samples were collected from the tail vein of the rats by the same manner as in Example 1, and the blood pH was measured using an i-stat analyzer as a blood gas analyzer (5 rats for each). As a control, the blood pH of the rats (10 rats) before induction of febrile seizures was measured. As a result, it was confirmed that the increase in the concentration of carbon dioxide to 10% (v/v) reduced the blood pH of the rats (FIG. 14).

5) Evaluation of Seizure

Electroencephalograms of the frontal lobe cortex and the occipital lobe cortex of the rats were measured by the same manner as in Example 1. An example of the data is illustrated. As a result, it was confirmed that epileptiform discharges, that is, duration of a spike could be suppressed significantly by changing the concentration of carbon dioxide to 10% (v/v) (FIG. 15). Results of seizure duration (second) are illustrated (FIG. 16). It was confirmed that a seizure could be suppressed significantly by changing the concentration of carbon dioxide to 10% (v/v).

6) Result

As described above, it was confirmed that, even in Kyo811 rats as a more severe epileptic model, the blood pH was reduced by the administration of carbon dioxide, and that electroencephalograms were improved.

### Reference Literature 1

Tokuda S, Kuramoto T, Tanaka K, Kaneko S, Takeuchi IK, SasaM, Serikawa T. The ataxic groggy rat has a missense mutation in the P/Q-type voltage-gated Ca2+ channel alphalA subunit gene and exhibits absence seizures. BRAINRESEARCH 1133 (2007) 168-177

### Reference Literature 2

Tanaka K, Shirakawa H, Okada K, Konno M, Nakagawa T, Serikawa T, Kaneko S. Increased Ca2+ channel currents in cerebellar Purkinje cells of the ataxic groggy rat. Neuroscience Letters 426 (2007) 75-80

### Reference Literature 3

Ohmori I, Ouchida M, Ohtsuka Y, Oka E, Shimizu K. Significant correlation of the SCN1A mutations and severe myoclonic epilepsy in infancy. Biochem Biophys Res Commun. 295(1), 17-23 (2002).

### Reference Literature 4

Escagy A, Heils A, MacDonald B T, Haug K, Sander T, Meisler M H. A novel SCN1A mutation associated with generalized epilepsy with febrile seizure plus—and prevalence of variants in patients with epilepsy. Am J Hum Genet 68: 866-873 (2001).

### Reference Literature 5

Mashimo T, Ohmori I, Ouchida M, Ohno Y, Tsurumi T, Miki T, Wakamori M, Ishihara S, Yoshida T, Takizawa A, Kato M, Hirabayashi M, Sasa M, Mori Y, Serikawa T. A missense mutation of the gene encoding voltage-dependent sodium channel (Nav1.1) confers susceptibility to febrile seizures in rats. J Neurosci. 30(16):5744-5753 (2010).

### INDUSTRIAL APPLICABILITY

As described in detail above, in the present invention, by placing the gas bottle, the device, or the like for a therapy of a disease accompanied by epileptiform discharges at homes or educational institutions or by carrying a small gas bottle, when a disease accompanied by epileptiform discharges occur, it is possible to easily reduce a symptom such as a seizure accompanied by the disease by inhaling, from the bottle, a therapeutic agent containing carbon dioxide as an active ingredient, specifically carbon dioxide gas.

In addition, by development of a gas bottle or an inhalation gas device using the therapeutic agent of the present invention for a disease accompanied by epileptiform discharges, the following effects are expected.

(1) An effect of suppressing a seizure is fast-acting.

(2) In a case of an unpredictable seizure at home or the like, surrounding persons (family members or the like) can cope therewith easily.

(3) Use thereof is simple.

(4) It is inexpensive.

(5) In status epilepticus, it is necessary to administer a medical substance intravenously. A fast-acting medication can be performed to a patient whose intravenous line is not easily ensured (such as children).

(6) When an epilepsy syndrome which is not easily suppressed with conventional medical substances is treated or status epilepticus is treated in an intensive care unit, it is possible to suppress a seizure while breathing is completely controlled.

Due to the present invention, it is possible to expect an incomparably better therapy which is applicable immediately upon a seizure and has a fast-acting property than in administration of a conventional therapeutic agent. Particularly, many of the epileptic patients are children, and therefore it is expected to make a system in which a handy inhalation gas (bottle) is placed at each home to be able to cope with a seizure immediately. It is said that about one million epileptic patients are present in Japan. The therapeutic agent of the present invention can act effectively not only against epilepsy but also a disease accompanied by epileptiform discharges. Therefore, a larger market therefor is expected.

1. An inhalation gas device for a therapy of a disease accompanied by epileptiform discharges comprising a medical gas bottle, and a medical inhalation gas device connected to the medical gas bottle, the inhalation gas device for a therapy of a disease accompanied by epileptiform discharges including the following 1) and 2):
1) a therapeutic agent for a disease accompanied by epileptiform discharges containing carbon dioxide as an active ingredient being filled in the medical gas bottle; and
2) the medical inhalation gas device being provided with a gas inhalation mask.

2. The inhalation gas device for a therapy of a disease accompanied by epileptiform discharges according to claim 1, wherein the medical inhalation gas device of 2) includes a gas extraction member which is attached to an opening portion of the medical gas bottle of 1) and discharges the therapeutic agent for a disease filled in the medical gas bottle from a discharge nozzle.

3. The inhalation gas device for a therapy of a disease accompanied by epileptiform discharges according to claim 2, wherein the gas inhalation mask provided in the medical inhalation gas device of 2) is a gas inhalation mask which has a base side connecting portion in which a mask base portion side is connected to the discharge nozzle of the medical inhalation gas device of 2), an inhalation shape portion provided on a mask distal end side having a shape in which a distal end edge covers a periphery of a mouth and a nose of an inhaler when used, and an enlarged portion which connects the base side connecting portion and the inhalation shape portion.

4. The inhalation gas device for a therapy of a disease according to claim 3, wherein the entire gas inhalation mask is formed of a soft material through which no gas permeates, and a peripheral edge gas introducing portion is provided on a distal end portion peripheral edge of the inhalation shape portion, the peripheral edge gas introducing portion expanding by introduction of the therapeutic agent for a disease discharged from the discharge nozzle to have a shape of the inhalation shape portion, and being contractible when the therapeutic agent for a disease is not introduced,
a resistance member which gives resistance against flow of the therapeutic agent for a disease from the discharge nozzle toward the inhalation shape portion is provided on the base side connecting portion, and
a gas guide flow path, which introduces the therapeutic agent for a disease discharged from the discharge nozzle into the peripheral edge gas introducing portion, branches from a space between the resistance member and the discharge nozzle in the base side connecting portion.

5. The inhalation gas device for a therapy of a disease according to claim 4, wherein the resistance member is a sintered filter, an orifice, or a valve.

6. The inhalation gas device for a therapy of a disease according to claim 4, wherein the gas guide flow path is integrally formed with the enlarged portion.

7. The inhalation gas device for a therapy of a disease according to claim 3, wherein the entire gas inhalation mask is formed of a soft material through which no gas permeates, and a peripheral edge gas introducing portion is provided on a distal end portion peripheral edge of the inhalation shape portion, the peripheral edge gas introducing portion expanding by introduction of the therapeutic agent for a disease discharged from the discharge nozzle to have a shape of the inhalation shape portion, and being contractible when the therapeutic agent for a disease is not introduced,
a shielding member which shields flow of the therapeutic agent for a disease from the discharge nozzle toward the inhalation shape portion is provided in the base side connecting portion,
a gas guide flow path, which introduces the therapeutic agent for a disease discharged from the discharge nozzle into the peripheral edge gas introducing portion, branches from a space between the shielding member and the discharge nozzle in the base side connecting portion, and an inhalation gas discharge portion which discharges the therapeutic agent for a disease toward inside of the enlarged portion is provided in at least one of the peripheral edge gas introducing portion and the gas guide flow path.

8. The inhalation gas device for a therapy of a disease according to claim 7, wherein the gas guide flow path is integrally formed with the enlarged portion.

9. The inhalation gas device for a therapy of a disease according to claim 1, wherein the disease accompanied by epileptiform discharges is epilepsy.

10. The inhalation gas device for a therapy of a disease according to claim 1, wherein the therapeutic agent for a disease accompanied by epileptiform discharges containing carbon dioxide as an active ingredient is an inhalation therapeutic agent.

11. The inhalation gas device for a therapy of a disease according to claim 10, wherein the medical gas bottle is filled with a therapeutic agent for a disease so that a concentration of carbon dioxide in the inhaled gas at the time of inhalation is 1 to 10% (v/v).
